(19) United States
(12) Patent Application Publication
(10) Pub. No.: US 2026/0223978 A1
(43) Pub. Date: Aug. 6, 2026

(54) SENSORIZED INSOLE FOR SHOES

(71) Applicant: FONDAZIONE ISTITUTO ITALIANO DI TECNOLOGIA, Genova (IT)

(72) Inventors: Gianluca MILANI, Genova (IT); Enrico VALLI, Genova (IT); Simone DUSSONI, Genova (IT); Maurizio BORGAGNI, Genova (IT); Daniele PUCCI, Genova (IT); Marco MAGGIALI, Genova (IT)

(73) Assignee: FONDAZIONE ISTITUTO ITALIANO DI TECNOLOGIA, Genova (IT)

(21) Appl. No.: 19/136,375

(22) PCT Filed: Nov. 21, 2023
(86) PCT No.: PCT/IB2023/061721

§ 371 Date: Jun. 6, 2025

(30) Foreign Application Priority Data
Dec. 12, 2022 (IT) 102022000025371

(51) Int. Cl. A43B 3/44; A61B 5/00; A61B 5/11

(52) U.S. Cl. CPC A43B 3/44; A61B 5/1116; A61B 5/112; A61B 5/6807; A61B 5/7275

(57) ABSTRACT

A sensorized insole for being placed on a shoe insole, including a sensor plane, a dielectric layer, and a ground layer. The dielectric layer separates the ground layer from the sensor plane. In use a compression of the dielectric layer causes a capacitance variation between the ground layer and at least one of a plurality of the sensor pads formed in a sensor layer of the sensor plane. Each sensor pad is electrically connected to a respective electrical terminal by a track of conductive material. The sensor layer also includes a shielding layer separated from the sensor layer by a support layer of flexible material. The sensor plane is arranged such that the shielding layer is interposed between the sensor layer and the dielectric layer. The shielding layer includes a plurality of through holes, a plurality of first shielding elements and a plurality of second shielding elements.

### TECHNICAL FIELD

The present invention concerns a sensorized insole for shoes.

### BACKGROUND

Gait and posture analysis are very important in the health sector because of the close relationship between gait and posture with certain diseases and their progression. For example, the real-time control of a Parkinson's patient's gait allows monitoring the progress of the disease.

In addition, gait analysis has become an important aspect in the sports sector, particularly in competitive sports. In fact, a gait analysis makes it possible to correct any defects in the athlete's gait, improving the performance thereof.

In order to provide information on a subject's gait, several solutions involving sensorized insoles have been proposed in the literature.

A first example of sensorized insole is known from the article [1], by Gonzàlez et al. In this solution, four Force Sensitive Resistors (FSR) are applied on the lower surface of a conventional insole. Each of the four FSR sensors has an elongated structure to the ends of which electrical wires of a flat cable are soldered. The flat cable is in turn connected to an Inertial Measurement Unit (IMU) which analyses the data and transmits them to a mobile device via a Bluetooth connection.

Another example of sensorized insole is known from the article [2], by Oerbekke and et al. This article describes the OpenGo sensorized insole, marketed by the German company Moticon. The insole consists of several superimposed layers. Going in order from top to bottom, there are an upper layer, a ground layer, a dielectric layer, a sensor support layer, and a lower layer with a slot into which an electronic module is inserted that can read the data of the sensors and transmit them to a mobile device application, such as for example a smartphone. The article does not deal with the electrical connections between electronics and sensors.

From the article [3] a sensorized, flexible shoe insole is known. The insole has a piezoelectric polyvinylidene fluoride (PVDF) layer on which 24 square copper electrodes etched onto two polyimide substrates that sandwich the PVDF layer are positioned. To protect the electrodes, two outer layers of polyethylene terephthalate (PET) are provided.

The polyimide layers that house the sensors and the electrical traces of the insole are flexible, but not stretchable, which is a limitation when the material is used in an insole and subject to significant stresses due to walking.

Furthermore, this solution has the limitation that the piezoelectric sensors used do not have the ability to measure with precision the pressure under static conditions. In detail, the piezoelectric sensors use materials that can generate an electrical charge on the surface if subjected to pressure. However, this charge dissipates over time, which means that, as the time passes by, the sensor loses its ability to measure static pressure. Although the pressure on the insole is a quantity that varies continuously during dynamic activities such as walking, in some situations (e.g. check of a subject's ability to stand still) it might be useful to measure static pressure over time; something that would not be possible with piezoelectric technology.

From the article [4] a flexible capacitive sensor suitable for being inserted into an insole for continuous analysis of a walking signal is known. This article, however, does not explain how to integrate the signal acquisition electronics into the insole. The sensor is made by using a flexible, but not stretchable material, which could lead to robustness problems when highly stressed. The sensor also has a very low signal acquisition frequency (approximately 0.1-0.5 Hz). In consideration of this, the device presented in the article can be used for calorie estimation as proposed by the authors but does not make it suitable for monitoring the pressure distribution during walking activity.

In addition to what is reported above, the signals generated by the sensors of the proposed sensorized insoles are perturbed by electronic noise, in particular due to the operation of the other sensors and/or of the electrical tracks of the sensorized insole as well as to any sources of electromagnetic radiations external to the sensorized insole—for example, personal portable devices capable of generating electromagnetic waves such as smartphones and/or wearables carried or worn by a user.

The electronic noise overlap the useful signal generated by the sensors, reducing the overall sensitivity of the sensorized insole, limiting the useful information that can be extracted from the signals generated by the sensorized insole and, consequently, the analysis of the user's gait based on the signals generated by the sensorized insole.

### REFERENCES

[1] I. Gonzalez and et al., “An Ambulatory System for Gait Monitoring Based on Wireless Sensorized Insoles,” Sensors, vol. 15, pp. 16589-16613, 2015.
[2]

M. S. Oerbekke and et al., “Concurrent validity and reliability of wireless instrumented insoles measuring postural balance and temporal gait parameters,” Gait & Posture, vol. 51, pp. 116-124, January 2017.
[3] J. Chen and et al., “A Piezoelectric Flexible Insole System for Gait Monitoring of Internet of Heath Things,” IEEE International Conference on Flexible and Printable Sensors and Systems (FLEPS), 2020.
[4] Seong Won Park and et al., “Development of wearable and flexible insole type capacitive pressure sensor,” Organic Electronics, no. 53, pp. 213-220, 2018.

### SUMMARY

Aim of the present invention is to overcome the drawbacks of the prior art.

In particular, aim of the present invention is to present an electronically noise-resistant sensorized insole.

Furthermore, aim of the present invention is to present an insole capable of withstanding the mechanical stresses associated with the action of walking, running, standing, etc. performed by a user of the insole.

It is also an aim of the present invention to present a system comprising a sensorized insole that allows an accurate analysis of the gait, joint stress and estimation of the physical exertion performed by the user.

These and other aims of the present invention are achieved by a sensorized insole incorporating the features of the accompanying claims, which form an integral part of the present description.

A first aspect of the present invention concerns a sensorized insole, suitable for being placed on a shoe insole. The sensorized insole comprises a sensor plane, a dielectric layer, and a ground layer. The dielectric layer separates the ground layer from the sensor plane. In use a compression of the dielectric layer causes a capacitance variation between the ground layer and at least one of a plurality of the sensor pads formed in a sensor layer of the sensor plane. Each sensor pad is electrically connected to a respective electrical terminal by means of a track, trace, of conductive material.

The sensor plane further comprises a shielding layer separated from the sensor layer by a support layer made of flexible material. The sensor plane is arranged such that the shielding layer is interposed between the sensor layer and the dielectric layer.

The shielding layer comprises a plurality of through holes, a plurality of first shielding elements and a plurality of second shielding elements. Each through hole has a shape and size substantially corresponding to a respective sensor pad and is superimposed in plan view above the corresponding sensor pad. Each first shielding element surrounds a respective through hole. Finally, each second shielding element connects a respective first shielding element to an electrical terminal and is superimposed in plan view above a corresponding track.

Preferably, the second shielding elements and the tracks have a meandering shape. For example, the second shielding elements and the tracks are strips of metallic material, e.g. copper, comprising a plurality of meanders.

Thanks to the structure of the sensor plane described above, the sensorized insole is able to generate a reliable signal without affecting the posture and gait of a user using the insole in a shoe.

In particular, the shielding layer of the sensorized insole effectively shields electromagnetic radiations that hit the sensorized insole or from electromagnetic interferences generated by capacitance variations associated with adjacent pads, thereby preventing the signals generated by the insole from being buried by electronic noise. Furthermore, the structure of the shielding layer of the insole according to the present invention allows achieving this electronic noise resistance without limiting foot mobility or reducing user comfort. In fact, the shielding layer comprises a limited number of metal traces that do not hinder the deformation of the insole and, consequently, the movement of the user's foot.

In one embodiment, the sensor layer also comprises a plurality of third shielding elements. Each of these third shielding elements surrounds a respective sensor pad. Preferably, each third shielding element is superimposed in plan view above a corresponding first shielding element of the shielding layer and, even more preferably, has a shape and size corresponding thereto.

In addition, fourth shielding elements connect a respective third shielding element to an electrical shielding terminal. Advantageously, pairs of fourth shielding elements are placed on opposite sides of at least one track of conductive material.

Thanks to these features, the signal produced by the insole can be shielded even more effectively from both external and internal electromagnetic interferences.

In one embodiment, the sensor layer comprises a plurality of conductive lines electrically connected to the plurality of second and fourth shielding elements. Advantageously, the plurality of conductive lines forms-optionally together with the plurality of third and fourth shielding elements—a plurality of meshes on at least a portion of the sensor layer surface.

Additionally or alternatively, the shielding layer comprises a plurality of conductive lines electrically connected to the plurality of first and second shielding elements. Advantageously, the plurality of conductive lines forms a plurality of meshes on at least a portion of the surface of the shielding layer—optionally, the meshes are formed by one or more conductive lines and one or more of the plurality of first and/or second shielding elements.

Mesh formation by means of the conductive tracks makes it possible to significantly increase the resistance to electromagnetic disturbances without compromising the deformability of the insole.

In one embodiment, the sensor layer further comprises a first perimeter conductive line, a second perimeter conductive line, a third perimeter conductive line and a plurality of further conductive lines. The first conductive perimeter line surrounds at least part of the sensor pads and is electrically connected to the plurality of conductive lines and to the electrical shielding terminal. The second and third perimeter conductive lines are essentially parallel to the first perimeter conductive line and the third perimeter conductive line surrounds the second perimeter conductive line, which in turn surrounds the first perimeter conductive line. Both the second and third perimeter conductor lines are electrically connected to a reference terminal. In addition, the further conductive lines of the sensor layer are electrically connected to the second and third perimeter conductive lines and form—optionally, with the second and third perimeter conductive lines—a plurality of meshes therebetween.

Additionally or alternatively, the shielding layer comprises a fourth perimeter conductive line, a fifth perimeter conductive line, a sixth perimeter conductive line and a plurality of further conductive lines. The fourth conductive perimeter line surrounds at least part of the through holes and is electrically connected to the plurality of conductive lines and to the respective electrical shielding terminal. The fifth and sixth perimeter conductive lines are essentially parallel to the fourth perimeter conductive line. Similar to what is described above, the sixth perimeter conductive line surrounds the fifth perimeter conductive line, which in turn surrounds the fourth perimeter conductive line. Both the fifth perimeter conductive line and the sixth perimeter conductive line are electrically connected to a reference terminal. In addition, the further conductive lines of the shielding layer are electrically connected to the fifth and sixth perimeter conductive lines, and form—optionally with fifth and sixth perimeter conductive lines—a plurality of meshes therebetween.

Preferably, the perimeter conductive lines and/or the conductive lines of the sensor layer and of the shielding layer are formed so that they are superimposed in plan view.

The addition of one or both of these structures connected to a reference terminal makes it possible to further increase the robustness to the electronic noise inside or outside the sensorized insole, without limiting the deformability of the sensorized insole.

In one embodiment, the sensorized insole comprises more than one sensor layer and a corresponding shielding layer. Advantageously, the sensor pads of each sensor layer—and, consequently, the through holes of the corresponding shielding layer—are arranged in different positions of the insole—in plan view.

This multilayer structure makes it possible to significantly increase the number of measurements performed by the insole, without compromising its deformability or resistance to electronic noise.

A different aspect of the present invention concerns an analysis system. The analysis system comprises at least one sensorized insole according to any one of the embodiments described above and a processing system. In detail, the processing system comprises an acquisition module and a data processing module. The acquisition module is connected to each electrical terminal of the sensorized insole and is adapted to receive signals generated by the sensorized insole during its compression and to convert said signals into corresponding digital signals. Furthermore, the data processing module is adapted to receive the digital signals and process them to provide information on a posture and/or a gait of a corresponding user.

The analysis system allows for an accurate analysis of a user's posture and/or gait in an efficient manner. In particular, the quality of the signals-in terms of signal-to-noise ratio—provided by the sensorized insole allows to ease up the specifications of the acquisition module and the precision of the analyses performed. Advantageously, the deformability of the sensorized insole does not compromise the user's posture and/or gait which leads to the acquisition of unaltered data and, thus, to obtaining more reliable posture and/or gait analyses.

Further features and advantages of the present invention will be more evident from the description of the accompanying drawings.

### BRIEF DESCRIPTION OF THE DRAWINGS

The invention will be described hereinafter with reference to some examples, given by way of non-limiting example, and illustrated in the appended drawings.

In such drawings:

FIG. 1 is a schematic top view of a sensorized insole according to an embodiment of the present invention connected to a processing system;

FIG. 2 is a schematic side section of the sensorized insole of FIG. 1;

FIG. 3A is a top view of a sensor plane of the electrical circuit included in the sensorized insole of FIG. 1;

FIG. 3B is an enlarged view of a portion of the sensor plane in FIG. 3A included in box B indicated in FIG. 3A;

FIG. 3C is an enlarged view of a portion of the sensor plane in FIG. 3A included in box C indicated in FIG. 3A;

FIG. 4A is a top view of a shielding plane of the electrical circuit included in the sensorized insole of FIG. 1;

FIG. 4B is an enlarged view of a portion of the shielding plane in FIG. 4A included in box B indicated in FIG. 4A;

FIG. 5 is a top view of portions of meandering conductive traces used in the sensorized insole according to an embodiment of the present invention;

FIG. 6A is a top view of a part of a sensorized insole according to an alternative embodiment comprising two sensors levels, and

FIG. 6B is a qualitative exploded view of the sensor and shielding planes of the multilayer electrical circuit of the sensorized insole in FIG. 6A.

### DETAILED DESCRIPTION OF THE EMBODIMENTS

While the invention is susceptible to various alternative modifications, some preferred embodiments will be described in detail below. It must in any case be understood that there is no intention to limit the invention to the specific embodiment illustrated, but, on the contrary, the invention intends covering all the modifications and equivalents that fall within the scope of the invention as defined in the claims.

Unless otherwise defined, all the terms of the art, notations and other scientific terms used herein are intended to have the meanings commonly understood by those skilled in the art to which this description belongs. In some cases, terms with commonly understood meanings are defined herein for clarity's sake and/or ready reference; the insertion of such definitions in the present description must therefore not be interpreted as representative of a substantial difference with respect to what is generally understood in the art.

The terms “comprising”, “having”, “including” and “containing” are to be understood as open terms (i.e. the meaning “comprising, but not limited to”) and are to be considered as a support also for terms such as “essentially consist of”, “essentially consisting of”, “to consist of” or “consisting of”.

The use of “for example”, “etc.”, “or” indicates non-exclusive alternatives without limitation, unless otherwise indicated. The use of “includes” means “includes, but not limited to” unless otherwise indicated.

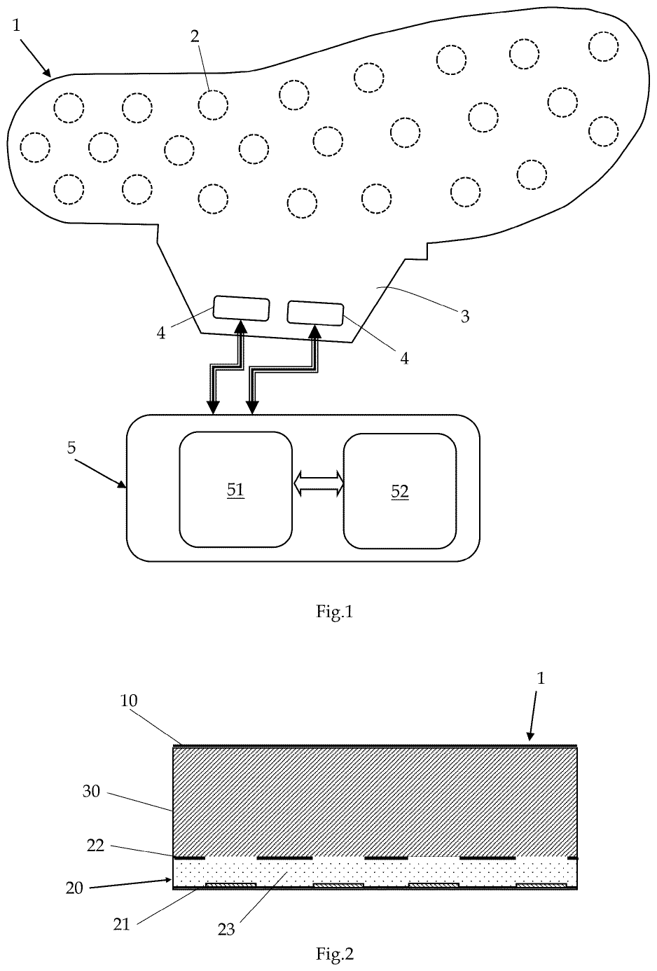

With reference to FIG. 1, a sensorized insole 1 according to an embodiment of the present invention is illustrated schematically.

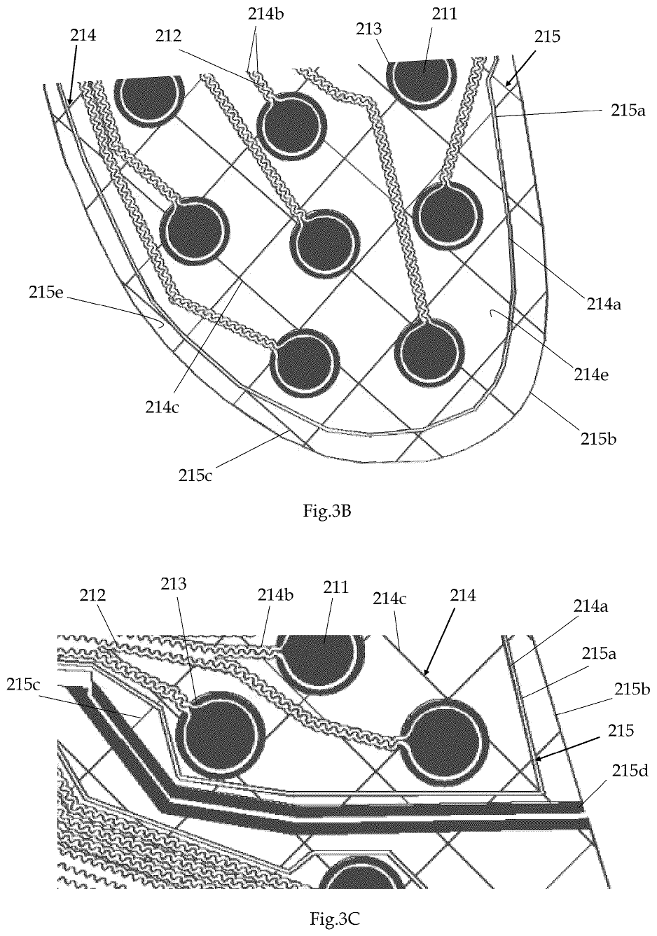

The sensorized insole 1 is realized as a multilayer element comprising a plurality of pressure sensors 2. The sensorized insole 1 a shape suitable to fit into a shoe, therefore it comprises an enlarged front area, intended to receive the forefoot, with respect to the rear area, intended to receive the heel. The pressure sensors 2 are electrically connected to a connector 4 formed in a side tab 3 of the sensorized insole 1. In the example in FIG. 1, two connectors 4 are formed in the side tab 3 of the insole 1. A subset of twelve pressure sensors 2 is connected to each connector via appropriate wirings, described below, and omitted for simplicity's sake in FIG. 1. Consequently, each connector comprises a total of fourteen pins (not illustrated in FIG. 1, but essentially corresponding to the terminals visible in FIG. 3A): one pin for each of the twelve pressure sensors 2 connected to the connector 4, one pin connected to a reference or ground voltage (ground pin) and one pin connected to a shielding voltage (shielding or shielding pin).

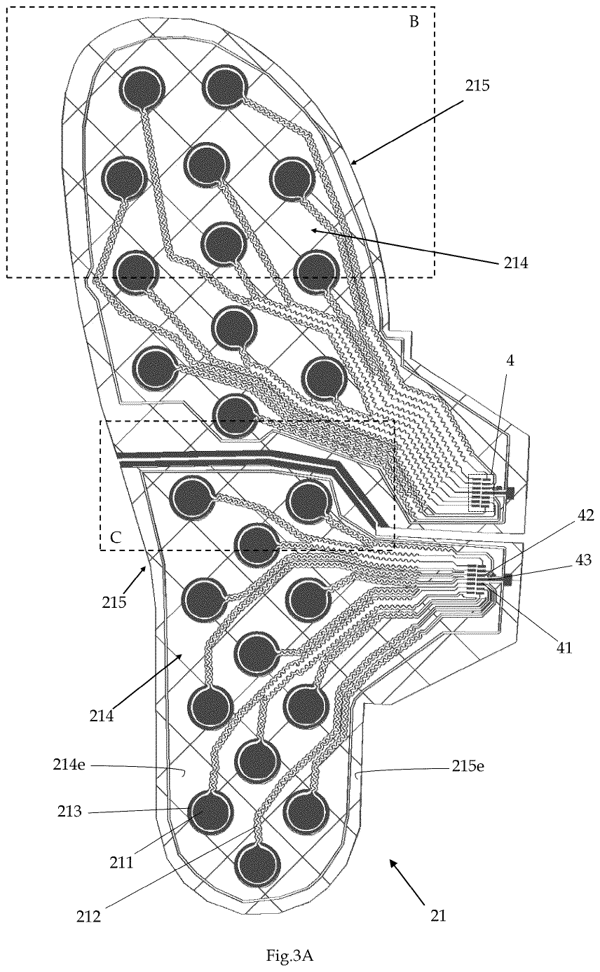

As illustrated in the schematic side section view in FIG. 2, the sensorized insole 1 comprises a ground layer 10 separated from a sensor plane 20 by an intermediate dielectric layer 30 made of dielectric material—e.g. TPU in the case under consideration. In the embodiment considered, the ground layer 10 comprises a conductive fabric sheet coupled to the dielectric layer 30. The dielectric layer 30 is a layer made of an electrically non-conductive material suitable for comfortably supporting the sole of a user's foot (not illustrated). Finally, the sensor plane 20 comprises a sensor layer 21 (lower) and a shielding layer 22 (upper) coupled with a support layer 23.

Preferably, the sensor plane 20 of the sensorized insole 1 is an extensible printed circuit board (PCT). Advantageously, the support layer 23 is made of flexible and extensible material, i.e. having the ability not only to flex but also to expand in the plane when subjected to a force transverse to the plane such as the weight of a human being.

For example, in a preferred embodiment, the support layer 23 is a polyether-based thermoplastic polyurethane (TPU) film, such as for example TFL-2EA, preferably less than 0.5 mm thick. TFL-2EA is soft to the touch and has an excellent ability to deform—in particular, to elongate—and tensile strength, it also has good microbiological and hydrolysis resistance, as well as wear resistance, making it a material suitable for the application.

The PCT of the sensor plane 20 is made in a known manner, i.e. each conductive layer—i.e. the sensor layer 21 and the shielding layer 22—are made starting from a respective copper sheet laminated to the support layer 23. The circuits of the layers are made by successive etching and removal of copper portions. Preferably, the sensors and the copper tracks of the sensor layer 21 are embedded in the support layer 23, thus ensuring high comfort during use of the sensorized insole by a user (not illustrated).

In the example considered, the sensor plane 20 is shaped to be deformable in all directions for substantially up to at least 30% of its size along the direction considered. Preferably, the sensor plane is made to withstand deformations that reach up to 50 % of its size along the direction considered.

As illustrated in FIG. 1, a processing system 5 connected to the connectors 4 is able to receive the signals generated by the pressure sensors 2 of the sensorized insole 1 during its use in order to process the information generated by the sensorized insole 1.

The processing system 5 comprises an acquisition module 51 and a data processing module. The acquisition module 51 receives the signals generated by the pressure sensors 2 and converts them into a digital signal, which the data processing module 52 then processes in a predefined manner in order to provide useful information for analysing the posture and/or the gait of a corresponding user (not illustrated).

For example, the acquisition module 51 comprises—in a non-limiting manner—one or more analogue-to-digital conversion (ADC) devices, filters, amplifiers, etc. Whereas, the processing module 52 comprises—in a non-limiting manner—one or more processors, DSPs, FPGAs, ASICs, etc. configured to perform the desired signal processing.

In the example considered, the acquisition module 51 comprises a pair of analogue/digital conversion devices (not illustrated), each connected to a respective connector 4 of the sensorized insole 1. The analogue/digital conversion devices are configured to convert signals generated by the pressure sensors 2 into digital signals and provide a reference voltage and a supply voltage to the sensorized insole 1.

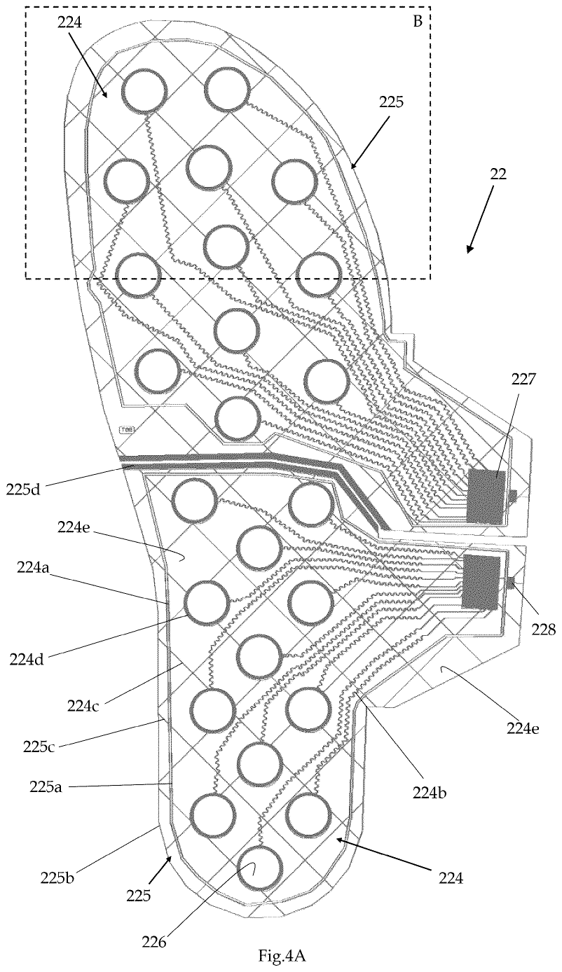

Returning to the sensorized insole 1, in the embodiment of the present invention, the sensor plane 20 comprises two superimposed layers: a sensor layer 21 (lower), illustrated schematically in FIGS. 3A, 3B and 3C, and a shielding layer 22 (upper), illustrated schematically in FIGS. 4A and 4B. The two layers are superimposed on each other, with the shielding layer 22 positioned above the sensor layer 21—that is, the shielding layer 22 is closer to the dielectric layer 30 than the sensor layer 21.

The sensor layer 21, illustrated in FIG. 3A and in the enlarged details in FIGS. 3B and 3C, comprises a plurality of the sensing elements, or sensor pads 211, of the pressure sensors 2.

In the example considered, the sensor layer 21 comprises a plurality of circular sensor pads 211, twenty-four in the example considered, each connected to a corresponding signal terminal, or pin, 41 of one of the two connectors 4 via a meandering-shaped conductive trace 212.

Preferably, the sensor layer 21 comprises a ring shielding 213 which surrounds a respective sensor pad 211. The ring shielding 213 is intended to provide enhanced protection from electromagnetic interferences where it is most needed—i.e. at the sensor pads 211. In particular, the ring shielding 213 avoids, or at least significantly attenuates, sensor-sensor cross-interferences.

Furthermore, in the embodiments of the present invention the sensor layer 21 comprises at least two voltage shielding elements, or AC shield 214, and two ground shielding elements, or GND shield 215.

In the exemplary embodiment of FIGS. 3A-3C, the GND shields 215 divide the sensor layer 21 into two portions. These two portions can be identified as a first portion, or front portion, which extends from a toe of the sensorized insole 1 up to approximately halfway the sensorized insole, and a second portion, or rear portion, which extends from a heel of the sensorized insole 1 up to approximately halfway the sensorized insole. Each GND shield 215 surrounds a respective AC shield 214, which in turn surrounds a respective subset of the sensor pads 211, twelve in the example considered.

As better visible in FIG. 3A and the detail in FIG. 3B, each AC shield 214 comprises a perimeter (conductive) line 214a, a plurality of insulation lines 214b and a plurality of grid lines 214c.

Specifically, the perimeter line 214a delimits the area of the AC shield 214 and is connected to a shielding terminal, or pin, 42 of the connector 4. Preferably, the shielding pin 42 is held at a shielding voltage by the processing system 5, so that the AC shield 214 is biased to the shielding voltage. Pairs of isolation lines 214b isolate respective conductive traces 212 from the AC shield 214—in other words, two isolation lines 214b surround a corresponding entire conductive trace 212 while remaining spaced apart from it. A pair of isolation lines 214b contains the respective conductive trace 212 from its connection to the respective sensor pad 211 to its connection to the shielding pin 42 of the connector 4.

Finally, the grid lines 214c extend in the space delimited by the perimeter line 214a, along two different directions transverse to each other, and are electrically connected to the perimeter line 214a, to the isolation lines 214c and to the shielding rings 213 that surround the sensor pads 211 comprised in the space delimited by the perimeter line 214a. In other words, the grid lines 214c form a mesh of the conductive lines delimited by the perimeter line 214a.

Each GND shield 215 surrounds a corresponding AC shield 214. The GND shield 215 comprises a first (conductive) perimeter line 215a (inner), a second perimeter line 215b (outer) and a plurality of grid lines 215c.

The first perimeter line 215a is substantially parallel to and spaced apart from the perimeter line 214a of the AC shield 214; furthermore, the first perimeter line 215a is connected to a reference terminal, or pin, 43 (e.g., a ground terminal) of the connector 4.

The second perimeter line 215b is a closed line formed by a first portion that basically follows a part of the perimeter of the sensorized insole 1 and a second portion that connects the two ends of the first portion between them; in addition, the second perimeter line 215b is connected to the ground pin of the connector 4.

In the exemplary embodiment of FIGS. 3A-3C, the second portions of the second perimeter lines 215b of the two GND shields 215 are parallel to each other and comprise a conductive strip 215d of substantially larger area than the remaining lines of the GND shields 215 and AC shield 214.

The conductive strips 215d act as a protective element against electrical discharges and electrically separate the circuits formed by the two different AC shields 214 and thus the two supply voltage signals provided by the analogue/digital converters of the acquisition module 51.

Finally, the grid lines 215c extend in the space between the first perimeter line 215a and the second perimeter line 215b, along two different directions transverse to each other, and are electrically connected to both perimeter lines 215a and 215b. In other words, the grid lines 215c form a mesh of conductive lines delimited by the first perimeter line 215a and by the second perimeter line 215b.

The shielding layer 22 also comprises at least two live shielding elements, or AC shield 224, and two ground shielding elements, or GND shield 225.

With reference to FIGS. 4A-4B, the GND shields 225 divide the shielding layer 22 into two portions. The two portions substantially correspond to the portions into which the sensor layer 21 is divided, i.e., a first portion, or front portion, extends from a front end of the sensorized insole 1 up to approximately halfway the sensorized insole and a second portion, or rear portion, extends from a rear end of the sensorized insole 1 up to approximately halfway the sensorized insole. Each GND shield 225 surrounds a respective AC shield 224, which in turn surrounds a respective subset of through holes 226, twelve in the example considered. The through holes 226 are formed in the shielding layer 22 so as to correspond to the sensor pads 211 on the sensor layer 21.

Each AC shield 224 comprises a perimeter (conductive) line 224a, a plurality of meander shielding lines 224b, a plurality of grid lines 224c and a plurality of ring shieldings 224d.

The perimeter line 224a delimits the area of the AC shield 224. Each shielding line 224b is shaped and arranged so as to be superimposed over a respective conductive trace 212 formed on the sensor layer 21 and is connected to a respective ring shielding 224d. Each ring shielding 224d surrounds a respective through hole 226. The perimeter line 224a and the shielding lines 224b are connected to a pad 227 which is connected to a shielding pin 42 of the connector 4 formed on the sensor layer 21 via a path (not illustrated). Finally, the grid lines 224c extend in the space delimited by the perimeter line 224a, along two different directions transverse to each other, and are electrically connected to the perimeter line 224a, to the shielding lines 224b and to the ring shieldings 226 comprised in the space delimited by the perimeter line 224a. In other words, the grid lines 224c form a mesh of conductive lines delimited by the perimeter line 224a.

Each GND shield 225 surrounds a corresponding AC shield 224. The GND shield 215 comprises a first (conductive) perimeter line 225a (inner), a second perimeter line 225b (outer) and a plurality of grid lines 225c.

The first perimeter line 225a is substantially parallel to and spaced apart from the perimeter line 224a of the AC shield 224; in addition, the first perimeter line 225a is connected to a pad 228 connected to the ground pin 43 of the connector 4 formed on the sensor layer 21 via a path.

The second perimeter line 225b is a closed line formed by a first portion that basically follows a part of the perimeter of the sensorized insole 1 and a second portion that connects the two ends of the first portion. Also the second perimeter line 225b is connected to the ground pin of the connector 4 via a path 228.

In the exemplary embodiment of FIGS. 4A-4B, the second portions of the second perimeter lines 225b of the two GND shields 225 are parallel to each other and comprise a conductive strip 225d of substantially larger area than the remaining lines of the GND shields 225 and AC shield 224. Preferably, each of the conductive strips is formed to correspond in size and arrangement to a respective conductive strip 215d of the GND shield 215 and AC shield 214 of the sensor layer.

Finally, the grid lines 225c extend in the space between the first perimeter line 225a and the second perimeter line 225b, along two different directions transverse to each other, and are electrically connected to both perimeter lines 225a and 225b. In other words, the grid lines 225c form a mesh of conductive lines delimited by the perimeter line 225a and by the second perimeter line 225b.

The Applicant has determined that the meshes defined by the grid lines 214c, 215c, 224c and 225c ensure an optimal electromagnetic shielding when they have a size (e.g., an area or a side) of the same order of magnitude as the sizes (e.g., an area or a diameter) of the sensors 2, or less than the same. Otherwise, there would be a negligible shielding effect and, in particular, the grids defined by the grid lines 214c and 224c would become too dense to have a sufficient number of electrical contacts with the rings 213 and 224d.

For example, in one embodiment, the mesh size defined by the grid lines 214c, 215c, 224c and 225c is about 2 cm on the side (i.e., square-shaped mesh) while the diameter of the sensors 2 is about 1.5 cm, with a total copper percentage over the total area of the sensor plane 20 around 1-2%.

For example, the conductive lines of the layers 21 and 22 are made of copper or aluminium. In one embodiment, the conductive lines of the layers 21 and 22 are made starting from a copper foil between 5 μm and 70 μm thick.

The structure of the layers 21 and 22, in particular, the conductive material grids formed by the grid lines 214c and 215c, and the ring shieldings 213 in the sensor layer 21, and the conductive lines 224c and 224d, and the ring shieldings 224d in the shielding layer 22 ensure a robust protection against electromagnetic interferences coming from both sources external to the sensorized insole 1 and from sources internal to the sensorized insole 1 (e.g. interferences between pressure sensors 2) without compromising flexibility and stretchability of the sensorized insole 1.

Preferably, the grid lines 214c and 215c, and the ring shieldings 213 in the sensor layer 21, and the conductive lines 224c and 225c in the shielding layer 22, and the ring shieldings 224d, respectively, are arranged so as to be substantially superimposed between them.

In the embodiment considered, in order to operate correctly during the extension and the change of length to which the sensorized insole 1 is subjected, the conductive traces 212 and the shielding lines 224b have a—corresponding—meandering shape.

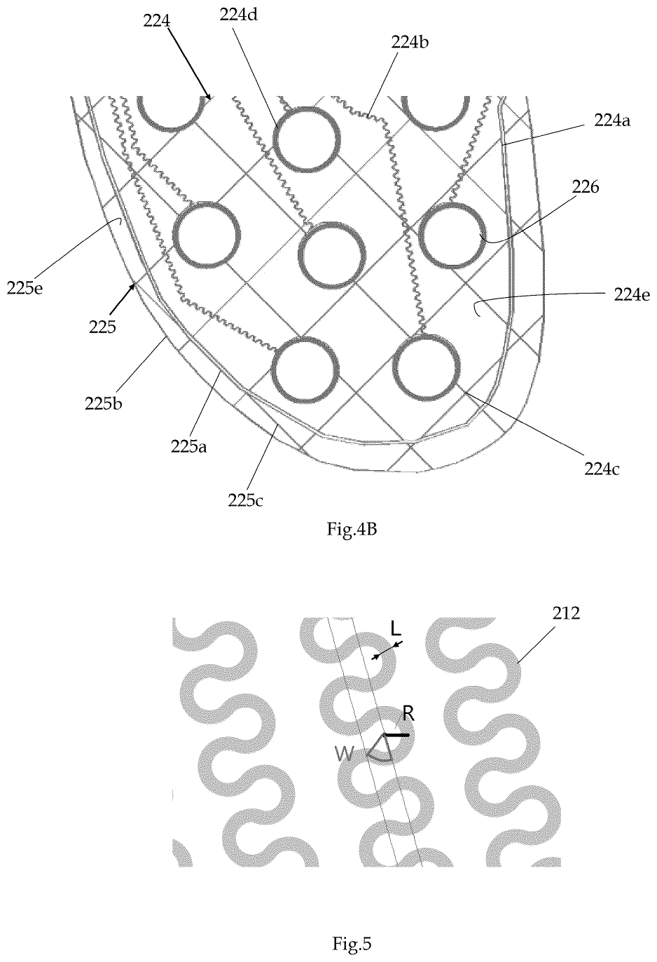

Preferably, the meanders of the conductive traces 212 and of the shielding lines have an essentially “horseshoe” or substantially semicircular shape-as shown in FIG. 5. The Applicant determined that the geometric parameters of the meanders that allow for optimal performance of the conductive traces 212 are as follows:
radius R, which must be greater than 0.125 mm and must not exceed the value of 0.5 cm—to ensure the passage between the sensor pads 211, i.e. that the traces 212 and the lines 224b do not come into contact with other conductive elements,
opening angles W comprised between −45° and +45°, and
width L of the trace between 0.1 mm and 0.3 mm.

The maximum possible repeatable elongation with this geometry is approximately 30% of the initial length. Furthermore, this shape of the conductive traces 212 has been demonstrated to withstand occasional events in which the deformation reaches up to 50 % of the initial length of the trace.

During operation, the compression of the dielectric layer 30 of the sensorized insole 1 during use by a user causes a variation in the distance between the ground layer 10 and the sensor plane 20, and thus in the capacitance of one or more sensors 2. The capacitance values associated with the sensors 2—in the example considered, the measurement of the capacitance associated with each sensor or its variation—are acquired and digitised by the acquisition module 51. For example, the connection between the analogue/digital converters of the acquisition module 51 and the respective pin 4 is made via a serial bus communication link. Each capacitive measurement is then converted by the processing module 52 into pressure values by means of a non-linear calibration. The calculated information, together with the position of the sensor 2, makes it possible to estimate the pressure distribution over the entire surface area of the sensorized insole 1 and, thus, of the user's insole.

However, it is clear that the above examples must not be interpreted in a limiting sense and the invention thus conceived is susceptible of numerous modifications and variations.

For example, in other embodiments (not illustrated), the sensorized insoles comprise multi-layers with more than two layers.

In particular, the low density of conductive material of the shielding layers according to the present invention can provide for one or more additional stacked formed sensor planes. Advantageously, each sensor plane provides sensors arranged in different positions than the positions of the sensors in the other sensor planes.

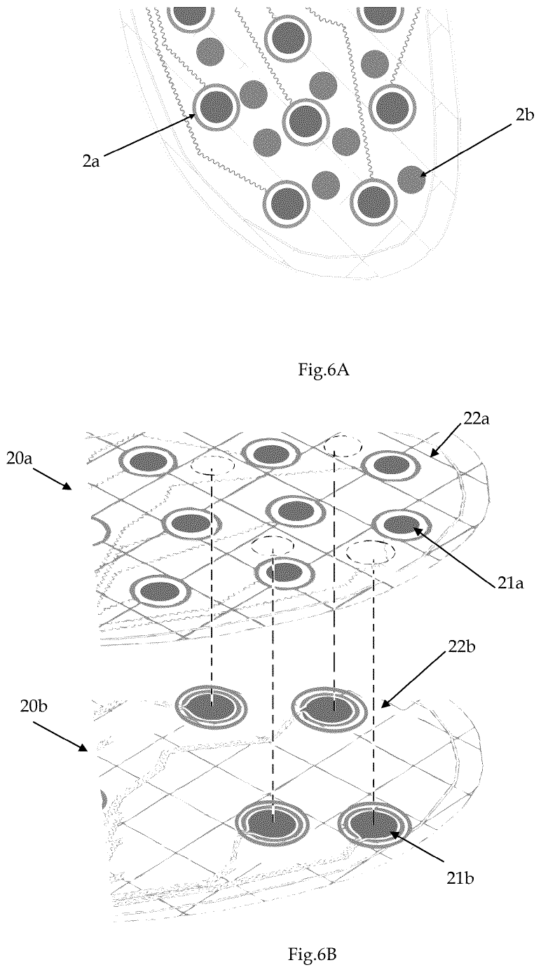

As illustrated schematically in FIGS. 6A and 6B, the alternative sensorized insole 1 comprises two superimposed sensor planes 20a and 20b, and the sensors 2a of the first sensor plane 20a have a different arrangement from the sensors 2b of the second sensor plane 20b.

Consequently, it is possible to realise sensorized insoles with optimised resolution and/or arrangement of the sensors according to the specific requirements of each application—e.g. medical, sports, etc.

Advantageously, the two sensor planes are independent of each other, each comprises a sensor layer 21a and 21b and a shielding layer 22a and 22b. In this way, the shielding layers are able to protect their sensors and traces also from the electromagnetic radiations generated by the other sensor layer.

In a simplified embodiment (not illustrated), the sensor layer and/or the shielding layer are without the grid lines. In this case, the electromagnetic shielding is limited to the shielding produced by the ring shieldings and by the meandering shieldings described above.

Naturally, all the details can be replaced with other technically-equivalent elements.

For example, the connecting traces may comprise meanders with a different shape from the horseshoe shape described above, e.g. in other embodiments (not illustrated) the meanders are in the shape of semi-circles, waves, rectangles, squares, etc.

In conclusion, the materials used, as well as the shapes and contingent dimensions of the devices, apparatuses and terminals mentioned above, may be any according to the specific implementation needs without thereby departing from the scope of protection of the following claims.

1-10. (canceled)

11. A sensorized insole configured to be placed on a shoe insole, the sensorized insole comprising: a sensor plane, a dielectric layer, and a ground layer, wherein:
the dielectric layer separates the ground layer from the sensor plane, in use a compression of the dielectric layer causes a capacitance variation between the ground layer and at least one of a plurality of sensor pads formed in a sensor layer of the sensor plane, and
each sensor pad is electrically connected to a respective electrical terminal by means of a track of conductive material,
wherein
the sensor plane further comprises a shielding layer separated from the sensor layer by a support layer made of flexible material, the sensor plane being arranged such that the shielding layer is interposed between the sensor layer and the dielectric layer, said shielding layer comprising:
a plurality of through holes, each through hole having a shape and size substantially corresponding to a respective sensor pad, and being superimposed in plan view above the corresponding sensor pad,
a plurality of first shielding elements, each first shielding element surrounding a respective through hole, and
a plurality of second shielding elements, each second shielding element connecting a respective first shielding element to an electrical shielding terminal, and being superimposed in plan view above a corresponding track, wherein the tracks and the second shielding elements have a corresponding meandering shape.

12. The sensorized insole according to claim 11, wherein the sensor layer further comprises a plurality of third shielding elements, each third shielding element surrounding a respective sensor pad, and being superimposed in plan view above a corresponding first shielding element of the shielding layer.

13. The sensorized insole according to claim 12, wherein the sensor layer further comprises fourth shielding elements, each fourth shielding element connecting a respective third shielding element to an electrical shield terminal, and wherein pairs of fourth shielding elements are placed on opposite sides of at least one track of conductive material.

14. The sensorized insole according to claim 13, wherein the sensor layer further comprises a plurality of conductive lines electrically connected to the plurality of third shielding elements and to the plurality of fourth elements shield, the plurality of conductive lines forming a plurality of meshes on at least a portion of the surface of the sensor layer.

15. The sensorized insole according to claim 14, wherein the shielding layer further comprises a plurality of conductive lines electrically connected to the plurality of first shielding elements and to the plurality of second shielding elements, the plurality of conductive lines forming a plurality of meshes on at least a portion of the surface of the shielding layer.

16. The sensorized insole according to claim 15, wherein the sensor layer further comprises:
a first perimeter conductive line, the perimeter conductive line surrounding at least part of the sensor pads and being electrically connected to the plurality of conductive lines and to the electric shielding terminal,
a second perimeter conductive line and a third perimeter conductive line substantially parallel to the first perimeter conductive line, wherein the second perimeter conductive line surrounds the first perimeter conductive line and the third perimeter conductive line surrounds the second perimeter line and both the second perimeter conductive line and the third perimeter conductive line are electrically connected to a reference terminal, and
a plurality of further conductive lines electrically connected to the second perimeter conductive line and to the third perimeter conductive line, the plurality of further conductive lines forming a plurality of meshes between the second line perimeter conductive line and the third perimeter conductive line.

17. The sensorized insole according to claim 16, wherein the shielding layer further comprises:
a fourth perimeter conductive line, the fourth perimeter conductive line surrounding at least part of the through holes and being electrically connected to the plurality of conductive lines and to the respective shielding electric terminal,
a fifth perimeter conductive line and a sixth perimeter conductive line substantially parallel to the fourth perimeter conductive line, wherein the fifth perimeter conductive line surrounds the fourth perimeter conductive line and the sixth perimeter conductive line surrounds the fifth perimeter line and both the fifth perimeter conductive line and the sixth perimeter conductive line are electrically connected to a reference terminal, and
a plurality of further conductive lines electrically connected to the fifth perimeter conductive line and to the sixth perimeter conductive line, the plurality of further conductive lines forming a plurality of meshes between the fifth perimeter conductor and the sixth perimeter conductor line.

18. The sensorized insole according to claim 17, wherein the fourth perimeter conductive line of the shielding layer is superimposed in plan view above the first perimeter conductive line of the sensor layer,
wherein the fifth perimeter conductive line of the shielding layer is superimposed in plan view above the second perimeter conductive line of the sensor layer, and
wherein the sixth perimeter conductive line of the shielding layer is superimposed in plan view above the third perimeter conductive line of the sensor layer.

19. The sensorized insole according to claim 11, further comprising at least one additional sensor layer and a corresponding at least one additional shielding layer, wherein the additional sensor layer and the additional shielding layer are shaped correspondingly to the sensor layer and the shielding layer, respectively, and
wherein the additional sensor pads of the additional sensor layer have a different arrangement than the sensor pads of the sensor layer, and the additional through holes of the additional shielding layer are arranged superimposed in plan view above the additional sensory pitches.

20. An system comprising at least a sensorized insole according to claim 11, and a processing system, wherein the processing system comprises:
an acquisition module connected to each electrical terminal of the sensorized insole and configured to receive signals generated by the sensorized insole during its compression and to convert said signals into corresponding digital signals, and
a data processing module suitable for receiving the digital signals and processing them to provide information on a posture and/or gait of a corresponding user.
